(19) United States
(12) Patent Application Publication
(10) Pub. No.: US 2021/0130807 A1
(43) Pub. Date: May 6, 2021

(54) KIT, SYSTEM, AND METHOD FOR NUCLEIC ACID EXTRACTION FROM SAMPLES

(71) Applicant: The Board of Regents of the University of Oklahoma, Norman, OK (US)

(72) Inventors: Vince A. Sandifer, Norman, OK (US); Kathleen E. Duncan, Norman, OK (US)

(21) Appl. No.: 17/141,909

(22) Filed: Jan. 5, 2021

Related U.S. Application Data
(63) Continuation-in-part of application No. PCT/US2020/023613, filed on Mar. 19, 2020.
(60) Provisional application No. 62/820,583, filed on Mar. 19, 2019.

(51) Int. Cl. C12N 15/10; C12Q 1/6806

(52) U.S. Cl. CPC C12N 15/1017; C12N 15/1006; C12Q 1/6806

(57) ABSTRACT

Kits, systems and methods for extracting nucleic acid, for example DNA or RNA, from a sample. The kits, systems and methods having a first syringe containing a lysis and preservative reagent, at least one second syringe containing an alcohol reagent, a third syringe containing a quantity of nucleic acid-soluble, nuclease-free water, a collection tube; and a filtering device.

### CROSS-REFERENCE TO RELATED APPLICATIONS

This application is a continuation-in-part of International Application No. PCT/US2020/023613, filed Mar. 19, 2020, which claims priority to U.S. Provisional Application No. 62/820,583, filed on Mar. 19, 2019, each of which is expressly incorporated herein by reference in its entirety.

### BACKGROUND

Microorganisms can have a positive, negative, or neutral effect in the environment. The presence of organisms in an environment can be of interest for many reasons. For example, when the environment is a source or supply of drinking water, detection of pathogenic organisms is desirable. When the environment is an oilfield or equipment used at an oilfield, detection of bacteria that influence corrosion and fouling is desirable. Detection of such organisms is frequently performed by analysis of nucleic acids, for example deoxyribonucleic acid (DNA) or ribonucleic acid (RNA) extracted from samples taken from such environments.

Nucleic acid extraction methods have traditionally been performed in a laboratory setting. Certain new methods have streamlined this process to allow for less equipment to be used, but some extra items such as a centrifuge or pipettes are still needed to complete the method. The Akonni TruTip method relies on a filter positioned inside a pipette tip to capture DNA. This method requires skill to perform and is not widely available. The MoBio Power Soil Kit enables nucleic acid extraction but requires extra equipment, such as pipettes and centrifuges. Current methods are designed for the user to return to a lab for extraction. A system and method enabling “on site” extraction of nucleic acid would be valuable.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein. The figures are not necessarily to scale and certain features and certain views of the figures may be shown as exaggerated in scale or in schematic in the interest of clarity and conciseness.

FIG. 1 is a perspective view one embodiment of a kit as described in the present disclosure.

FIG. 2 is a diagram showing how a filtering device of a kit of the present disclosure is detached from one syringe and reattached to another syringe via a connector.

FIG. 3 is a diagram showing two syringes attached to a filtering device by connecting a needle end of each syringe to an input end and an output end of the filtering device.

FIG. 4 is a perspective view of another embodiment of a kit used in Example 2.

FIG. 5 is a cross-sectional view of the filtering device shown in FIGS. 1 and 4.

FIG. 6 is a cross-sectional view of a syringe of the kit, the syringe containing extracted DNA attached to another syringe containing a detection reagent and/or DNA probe.

FIG. 7 is an elevational view showing one embodiment of a syringe of a kit of the present disclosure drawing a sample from a sample cup.

### DETAILED DESCRIPTION

The present disclosure is directed to kits, systems, and methods for the extraction and capture of nucleic acid from a sample. In particular, it enables a user to conduct nucleic acid extraction “on site” without requiring a sample to be transported back to a laboratory for the nucleic acid extraction. The kit, system, or method could be used in a laboratory setting as well. The kit and methods may be employed, in non-limiting examples, for extractions of environmental samples such as but not limited to oilfield equipment, oil and gas pipelines, oil holding tanks, oil storage tanks, wastewater, streams, rivers, bodies of water, soil, plants, or anywhere inside or outside of a laboratory setting. The kit could also be used for clinical samples such as but not limited to saliva, blood, tissue, urine, or fecal mater in or out of a laboratory setting. The kit, system, or method can be used for any solid or liquid sample, from which the user wants to extract nucleic acids. This novel method enables nucleic acid extraction to be optionally performed without using extra equipment such as a centrifuge, pipettes, or extra tubes, although these items can be used when desired to allow for easier extraction of difficult samples. Using different reagents specific to either DNA or RNA, the method can extract DNA independently of RNA or RNA independently of DNA. It is also possible extract both RNA and DNA by selecting appropriate reagents. The inventors are aware of no current nucleic acid extraction method which uses multiple syringes attached in succession to a filtering device to extract nucleic acids.

Before describing various embodiments of the embodiments of the present disclosure in more detail by way of exemplary description, examples, and results, it is to be understood that the embodiments of the present disclosure are not limited in application to the details of methods and apparatus as set forth in the following description. The embodiments of the present disclosure are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that certain embodiments of the present disclosure can be practiced without these specific details. In other instances, features which are well known to persons of ordinary skill in the art have not been described in detail to avoid unnecessary complication of the description.

Unless otherwise defined herein, scientific and technical terms used in connection with the embodiments of the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which embodiments of the present disclosure pertain. All patents, published patent applications, and non-patent publications referenced in any portion of this application are herein expressly incorporated by reference in their entirety to the same extent as if each individual patent or publication was specifically and individually indicated to be incorporated by reference.

While the methods and apparatus of the embodiments of the present disclosure have been described in terms of particular embodiments, it will be apparent to those of skill in the art that variations may be applied to the thereto and in the steps or in the sequence of steps of the methods described herein without departing from the spirit and scope of the inventive concepts. All such similar substitutes and modifications apparent to those of skilled in the art are deemed to be within the spirit and scope of the systems as defined herein.

As utilized in accordance with the methods and apparatus of the embodiments of the present disclosure, the following terms, unless otherwise indicated, shall be understood to have the following meanings.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” or “approximately” is used to indicate that a value includes the inherent variation of error. Further, in this detailed description, each numerical value (e.g., time or frequency) should be read once as modified by the term “about” (unless already expressly so modified), and then read again as not so modified unless otherwise indicated in context. The use of the term “about” or “approximately” may mean a range including ±0.5%, or ±1%, ±2%, or ±3%, or ±4%, or ±5%, ±6%, or ±7%, or ±8%, or ±9%, or ±10%, or ±11%, or ±12%, or ±13%, or ±14%, or ±15%, or ±25% of the subsequent number unless otherwise stated.

As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, or at least 90% of the time, or at least 95% of the time, or at least 98% of the time.

Features of any of the embodiments described herein may be combined with any of the other embodiments to create a new embodiment. As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50. Similarly, fractional amounts between any two consecutive integers are intended to be included herein, such as, but not limited to, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, and 0.95. For example, the range 3 to 4 includes, but is not limited to, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, and 3.95. Thus, even if specific data points within the range, or even no data points within the range, are explicitly identified or specifically referred to, it is to be understood that any data points within the range are to be considered to have been specified, and that the inventors possessed knowledge of the entire range and the points within the range. Reference to a series of ranges includes ranges which combine the values of the boundaries of different ranges within the series. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range.

Thus, to further illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure.

Where used herein the term “nucleic acid” is intended to refer to either deoxyribonucleic acid (DNA) or ribonucleic acid (RNA) or both DNA and RNA.

The inventive concepts of the present disclosure will be more readily understood by reference to the following examples and embodiments, which are included merely for purposes of illustration of certain aspects and embodiments thereof, and are not intended to be limitations of the disclosure in any way whatsoever. Those skilled in the art will promptly recognize appropriate variations of the apparatus, compositions, components, procedures and method shown below.

### Example 1

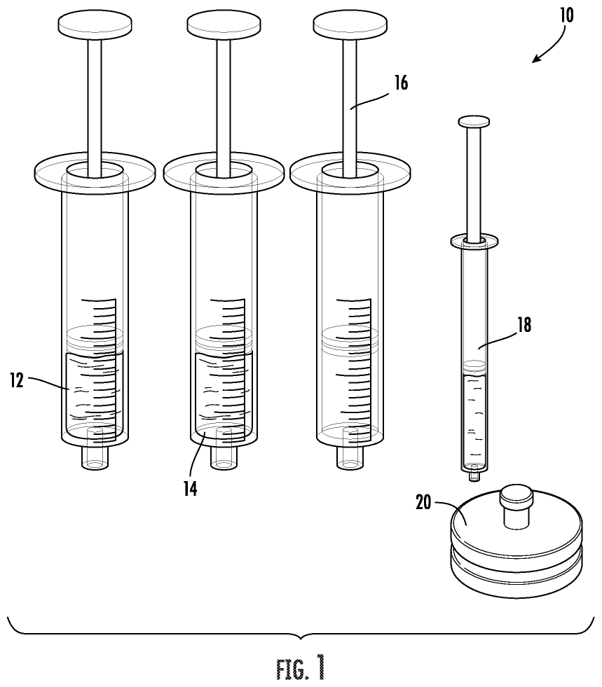

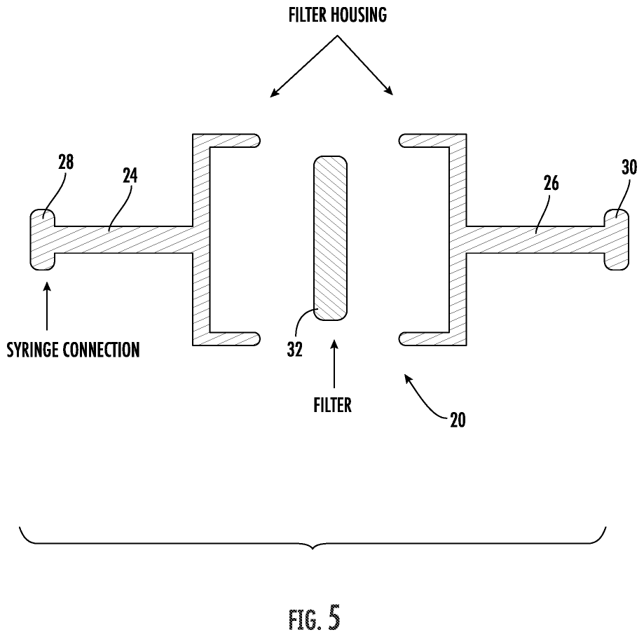

Referring now to FIG. 1, shown therein is one embodiment of a kit 10 provided with a plurality of syringes 12, 14, 16 and 18 and a filtering device 20. Syringe 12 contains a liquid containing a lysis and preservative reagent for extracting and preserving nucleic acids from living cells in a sample. Syringe 14 contains an alcohol reagent. Syringe 16, when included, is not prefilled with a solution. Syringe 18 contains nucleic acid-soluble, nuclease-free water. The filtering device 20 is a housing 22 constructed to have two opposing leur connectors 24 and 26 (e.g., leur lock or leur slip), one at an input end 28 and a second at an output end 30, and containing a filter 32 (e.g., glass microfibers, cellulose fiber, nylon fiber) (FIG. 5) therein which is able to capture DNA. The empty suringe 14 can be used instead of a separate syringe 16 for drying the filtering device 20.

Examples of the components of DNA lysis and preservative reagents that can be used herein include, but are not limited to, a buffer such as Tris-HCl (tris(hydroxymethyl)aminomethane, or HEPES(4-(2-hydroxyethyl)-1-piperazineethanesulfonic acid); a salt such as guanidine thiocyanate, NaCl, KCl, or (NH₄)₂SO₄; and a detergent such as CHAPS (3-[(3-cholamidopropyl)dimethylammonio]-1-propanesulfonate), sodium dodecyl sulfate (SDS), or ethyl trimethyl ammonium bromide. The lysis solution can optionally contain an alcohol such as ethanol or isopropanol. Lysis and preservative reagents that are selective for DNA (versus RNA) generally have a pH of about 8 or higher (e.g., to 12).

Examples of the components of RNA lysis and preservative reagents that can be used herein include, but are not limited to, a salt such as guanidine thiocyanate, NaCl, KCl, or (NH₄)₂SO₄; and a detergent such as CHAPS (3-[(3-cholamidopropyl)dimethylammonio]-1-propanesulfonate), sodium dodecyl sulfate (SDS), or ethyl trimethyl ammonium bromide; phenol chloroform. The lysis solution can optionally contain an alcohol such as ethanol or isopropanol. The solution may also contain protease inhibitors. Lysis and preservative reagents that are selective for RNA (versus DNA) generally have a pH of about 5 or lower (e.g., to 2.5), for example 4.7 or lower.

Examples of alcohol reagents include but are not limited to methanol, ethanol, and isopropanol, which can be used at various concentrations to optimize an extraction.

Examples of elution solutions that can be used include, but are not limited to, nuclease free water, that has been treated to ensure there are no nuclease enzymes, which can break the bonds of DNA. This elution solution could also contain Tris buffer.

Examples of filters that can be used include, but are not limited to, cellulose, glass fiber or polyethersulfone (PES) filters having pore sizes of 0.22 μm, 0.45 μm, 0.7 μm, and 1.0 μm.

Examples of syringes include, but are not limited to, those having volumes of 0.5 ml or larger, depending on the sample size input, for example, 5, 1, and 20 ml syringes (or larger can be used for the lysis, washing, and drying steps, for a 15 ml sample, then a 1 ml syringe can be used for elution. Syringes with larger volumes, such as 10 ml to 60 ml syringe can be used to collect larger samples for extraction. In one example, 2 smaller extractions can be performed then combined into one for DNA elution into 1 tube.

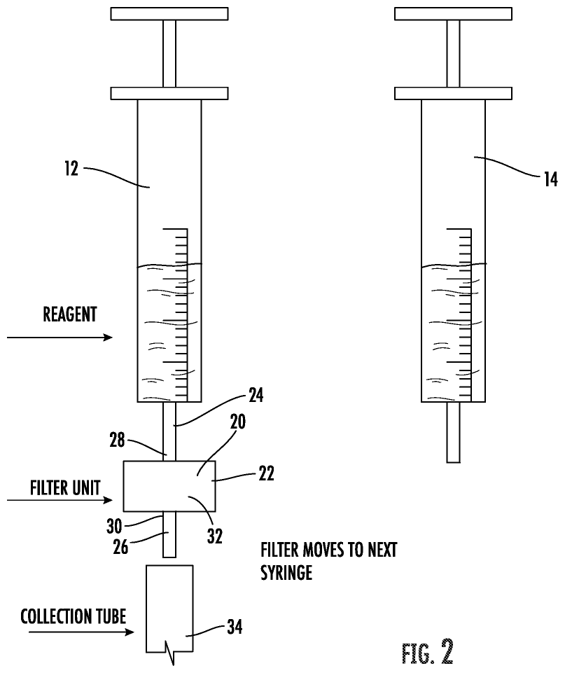

In a first step of the method, the sample to be analyzed is drawn into syringe 12 which is then capped. The liquid mixture containing the sample and the lysis and preservative reagent is shaken to mix the contents then allowed to stand for approximately 10 minutes (but could be longer or shorter time), causing lysis of the cells of the sample and extraction of nucleic acids therefrom. The input end 28 of the filtering device 20 is then attached to the needle end of syringe 12. The liquid mixture in syringe 12 is pushed into the filtering device 20 wherein the nucleic acids extracted from the cells in the sample are captured on the filter 32 as the liquid mixture passes through the filtering device 20 and out through the output end 30 of the filtering device 20. The nucleic acids are captured on and adhered to the microfiber filter 32 (FIG. 2).

In a second step, the filtering device 20 is then separated from syringe 12 and the input end 28 is attached to the needle end of syringe 14 (FIG. 2). As noted above, syringe 14 contains an alcohol reagent. The alcohol reagent causes precipitation of the nucleic acid on the filter 32 and causes rinsing of undesired compounds from the precipitated nucleic acid when the alcohol reagent is expressed from the syringe 14 into and through the filtering device 20.

In a third step, the filtering device 20 is then separated from syringe 14 and the input end 28 is attached to the needle end of syringe 16. As noted above, syringe 16 is not prefilled, and is used to push air through the filter 20 causing the filter 20 to be dried of the alcohol rinse residue remaining from the alcohol rinse of syringe 14. Alternately, as noted, in place of syringe 16, the empty syringe 14 can be used for this step. Using an empty syringe 16 for drying step simplifies decision-making and workflow by enabling the user to use separate syringes for each step rather than having to reuse syringe 14 for the drying step.

In a fourth step, the filtering device 20 is then separated from syringe 16 (or syringe 14 if it is used instead of a syringe 16) and the output end 30 is attached to the needle end of syringe 18 and the input end 28 is connected to a collection tube 34. Syringe 18 contains a quantity of nucleic acid-soluble, nuclease-free water and or buffer (e.g., Tris), which is then expressed into the filtering device 20 eluting the precipitated nucleic acids from the filter 32 and causing it to dissolve into the nuclease-free water and be rinsed from the filter 32 into the collection tube 34 for further treatment and/or analysis. Using these syringes in the sequence described herein, each step is followed by another, until completion of the nucleic acid extraction and rinsing process.

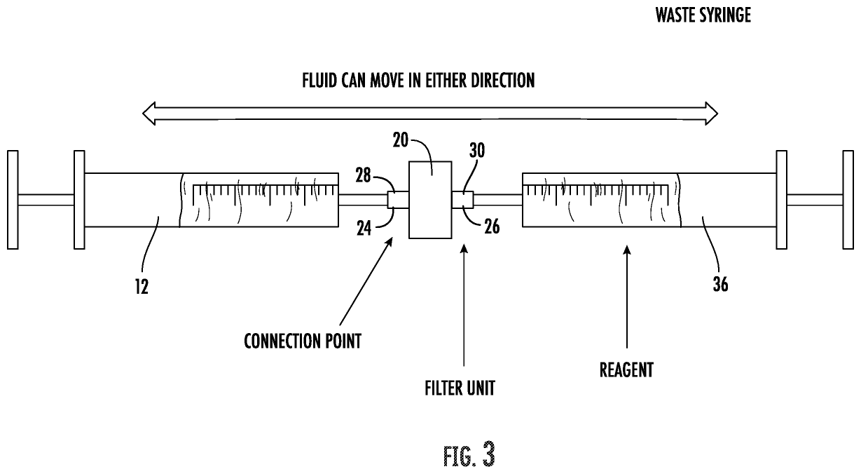

Any number of additional syringes could be used to vary the extraction process according to a user's needs. For example, a syringe with additional lysis and preservative reagent could be pushed through the filter 20 to lyse cells stuck in the filter 20 after syringe 12 is used. A second alcohol reagent syringe with a different concentration of alcohol or a different alcohol, e.g., isopropanol, could be used for optimization. A wash syringe could be filled with a salt reagent such as guanidine thiocyanate or guanidine HCl for use after lysis and before the alcohol wash syringe. This moving of a filter from one reagent dispenser (syringe) to another is a novel method for performing a DNA extraction. A second syringe 36 could be attached to the exit port 30 of the filter holder 20 (FIG. 3). The second syringe 36 can be used to hold filtered liquid expressed from a first syringe 12 which is then back-flushed through the filtering device 20 into the first syringe by pushing liquid back through the filtering device 20, for example to clear a clog in the filtering device 20 or in the first syringe 12. Each time the fluid passes through the filter 32, more nucleic acids are captured by the filter 32 during the lysis and washing steps. During the elution step each time the elution reagent passes through the filter 32 it dissolves more nucleic acid and increases the total nucleic acid yield in the collection tube 34. Thus, by pulling the liquid back into the syringe and pushing it through the filter two more times, yield can be enhanced.

### Example 2

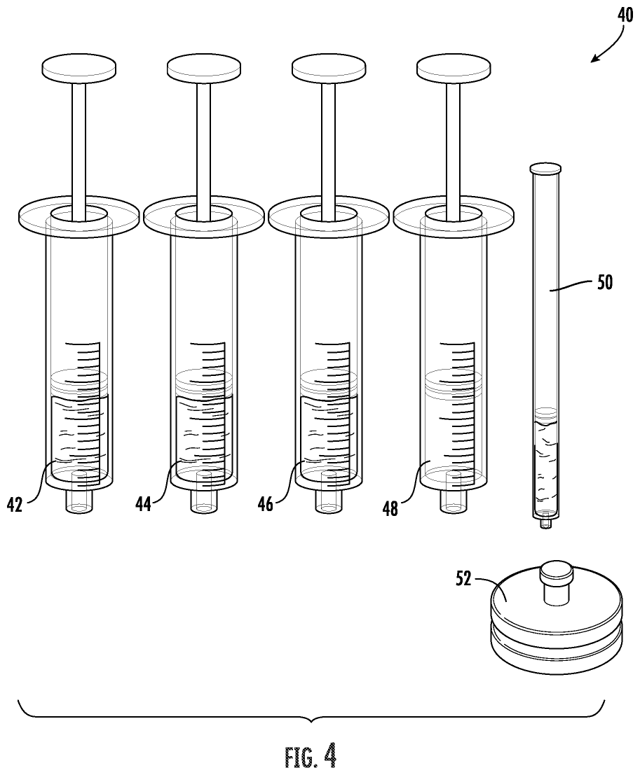

Referring now to FIG. 4, shown therein is another embodiment of a kit 40 provided with a plurality of syringes 42, 44, 46, 48 and 50 and a filtering device 52. The filtering device 52 is similar to the filtering device 20. Syringe 42 contains a liquid containing a lysis and preservative reagent for extracting nucleic acids from living cells in a sample, similar to syringe 12. Syringes 44 and 46 contain an alcohol reagent, similar to syringe 14. Syringe 48 is not prefilled with a solution, similar to syringe 16. Syringe 50 contains nucleic acid-soluble, nuclease-free water, similar to syringe 18.

In the first step of the method, the sample to be analyzed is drawn into syringe 42 which is then capped. The liquid mixture containing the sample and the lysis and preservative reagent is shaken to mix the contents then allowed to stand for approximately 10 minutes, causing lysis of the cells of the sample and extraction of nucleic acid therefrom. The input end of the filtering device 52 is then attached to the needle end of syringe 42. The liquid mixture in syringe 42 is pushed into the filtering device wherein the nucleic acid extracted from the cells in the sample is captured on the filter as the liquid mixture passes through the filtering device 52 and out through the output end of the filtering device 52. The nucleic acid is captured on and adheres to the filter.

In a second step, the filtering device 52 is then separated from syringe 42 and is attached to the needle end of syringe 44. As noted above, syringe 44 contains an alcohol reagent. The alcohol reagent causes precipitation of the nucleic acid on the filter and causes rinsing of undesired compounds from the precipitated nucleic acid when the alcohol reagent is expressed from the syringe 44 into and through the filtering device 52.

In a third step, the filtering device is then separated from syringe 44 and is attached to the needle end of syringe 46 which also contains an alcohol reagent for further rinsing the extracted nucleic acids.

In a fourth step, the filtering device is then separated from syringe 46 and is attached to the needle end of syringe 48. As noted above, syringe 48 is not prefilled, and is used to push air through the filter causing the filter to be dried of the alcohol rinse residue remaining from the alcohol rinse of syringe 46. Air from syringe 48 may be plunged 10 to 15 through the filtering device 52 to dry the nucleic acid sample. Before each subsequent plunging, the filtering device 52 is removed, the syringe plunger withdrawn to maximum empty volume, and the filtering device 52 reattached to the syringe. Alternatively, the empty syringe 46 can be used instead of the syringe 48.

After the final expression of air into the filtering device 52, the filtering device 52 is then separated from syringe 48 and the output end is attached to the needle end of syringe 50 and the input end is connected to a collection tube (not shown). Syringe 50 contains a quantity of nucleic acid-soluble, nuclease-free water which is then expressed into the filtering device 52 eluting the precipitated nucleic acid from the filter and causing it to be rinsed from the filter into the collection tube for further treatment and/or analysis.

### Example 3

In another embodiment, in a first step of the method, the sample to be analyzed is drawn into syringe 12 which is then capped. The liquid mixture containing the sample and the lysis and preservative reagent is shaken to mix the contents then allowed to stand for approximately 10 minutes or longer (days, weeks, months), causing lysis of the cells of the sample and extraction of nucleic acid therefrom. The input end of the filtering device 20 is then attached to the needle end of syringe 1. The liquid mixture in syringe 12 is pushed into the filtering device 20 wherein the nucleic acid extracted from the cells in the sample is captured on the filter as the liquid mixture passes through the filtering device 20 and out through the output end of the filtering device 20. The nucleic acid is captured on and adheres to the filtering device 20. To increase the nucleic acid capture, the fluid can be drawn into the syringe 12 by removing the filter and then reattaching and expressing the fluid through the filter again. This step can be repeated multiple times. Alternatively a syringe 36 could be attached to the exit port of the filter 20 (FIG. 3) and the reagent expressed through the filter into syringe 36. The the reagent from this syringe 36 can be expressed back through the filter into syringe 12. Repeating this step multiple times can increase nucleic acid yield.

In a second step, the filtering device 20 is then separated from syringe 12 and the input end is attached to the needle end of syringe 14. As noted above, syringe 14 contains an alcohol reagent. The alcohol reagent causes precipitation of the nucleic acid on the filter and causes rinsing of undesired compounds from the precipitated nucleic acid when the alcohol reagent is expressed from the syringe into and through the filtering device 20. Additionally a second syringe could be attached to the exit port of the filter (FIG. 3) and the reagent expressed through the filter into this second syringe. The reagent from this second syringe can be expressed through the filter into the first syringe. Repeating this step multiple times can increase nucleic acid yield. Allowing time between each reagent expression can increase nucleic acid precipitation, which will increase yield.

In a third step, the filtering device 20 is then separated from syringe 14 and the syringe is drawn to pull air into the syringe. Syringe 14 is then attached to the filter device 20 and the air is expressed into the filter to dry the filter. This step is repeated until no liquid exits the filter device. This use of Syringe 14 for drying is used in place of a Syringe 16 for drying.

In a fourth step, the filtering device 20 is then separated from syringe 14 and the output end is attached to the needle end of syringe 18 (Syringe 48 in other examples) and the input end is connected to a collection tube. Syringe 16 contains a quantity of nucleic acid-soluble, nuclease-free water and or buffer (i.e Tris), which is then expressed into the filtering device eluting the precipitated nucleic acid from the filter and causing it to be rinsed from the filter into the collection tube for further treatment and/or analysis. A second syringe can be attached to the exit port of the filtering device (FIG. 2) allowing for the liquid to be expressed through the filter and into this syringe. The elution reagent can then be expressed from this second syringe back into Syringe 16. Repeating this step multiple times can increase nucleic acid yield. Using these syringes in the sequence described herein, each step is followed by another, until completion of the nucleic acid extraction and rinsing process.

### Example 4

In place of collecting the nucleic acid into a tube during a final elution step, one or more further steps can be added for the detection of nucleic acid extracted.

For example, to elute the nucleic acid, a second syringe will be attached to the filter opposite the syringe filled with elution reagent (FIG. 3). The fluid will be cycled through the filter multiple times to dissolve the nucleic acid into the elution reagent. The total volume will then be pushed into one of the syringes and disconnected for use with the detection reagents.

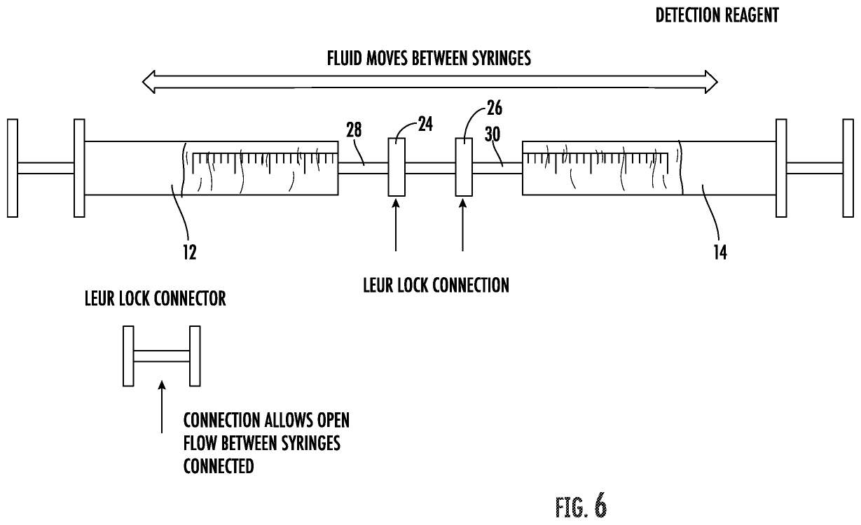

The syringe containing extracted nucleic acids can then be attached to a syringe containing detection reagents and/or nucleic acid probes (FIG. 6). The detection reagents are mixed with the nucleic acid by pushing the syringe contents from one syringe into another. The mixed solution can then be dispensed into a tube for analysis by instrument or visual inspection. The solution could also be directly read from the syringe if it is a colorimetric.

Examples of detection reagents include, but are not limited to, gold nanoparticles, silver nanoparticles, oligonucleotides and buffers such as TRIS. These nanoparticles and oligonucleotides can be modified with functional groups for targeted detection. In this example the reagents provide a visual detection using a colorimetric scale. However, other detection reagents used may require an additional instrument.

Advantages of the extraction methods described herein include but are not limited to, (1) it can be performed manually, i.e., extra equipment or power is not required, (2) it can be performed in any orientation, such as upside down or at an angle, (3) ability to easily move the filter device from one syringe to another, (4) the ability to adjust the volume of sample and/or prefilled syringe contents to be adjusted to meet the needs of a particular user, (5) the filtering device can be attached to a syringe at either end of the filtering device, enabling the filtering device or filter therein to be backwashed, for example to unclog filtering device or to increase nucleic acid yield.

### Example 5

It is possible to automate this extraction method by using a pump or similar device to push fluids across the filter. For example, the filter could be placed in a holder and then connected to a hose on both sides. These hoses would be connected to a tank of reagents and a pump to push them back and forth across the filter. First the the sample would be placed in a vessel with a lysis and preservative reagent, then connected to the pump and filter line. The sample would then be pumped across the filter to capture the nucleic acid. Next the vessel containing the lysis and preservative reagent would be disconnected and a wash reagent connected. This would be pumped across the filter to wash it. Next the wash reagent vessel would be disconnected and the pump used to push air across the filter to dry the filter. Next an elution reagent vessel would be attached and then the nucleic acid eluted from the filter and into the elution reagent vessel.

### Example 6

In all of the above examples the sample has the cell lysis step occur before filtering. Alternatively, the sample can be filtered prior to lysis by capturing the cells on the filter and then lysing them to capture the nucleic acid on the filter. For example, the sample could be drawn into a syringe and then connected to the filter. Once the sample is filtered the sample syringe is disconnected and a syringe containing lysis and preservative reagent is connected. Then a second syringe is connected to the other side of the filter. The lysis and preservative reagent is then pushed back and forth across the filter to capture the nucleic acid. The number of passes could be any number, such as but not limited to 1 to 1000 or more passes. The syringes are then disconnected. The filter is then connected to a wash reagent syringe and an empty syringe. The process of passing reagent across the filter is repeated. The number of passes could be any number, such as but not limited to 1 to 1000 or more passes. The nucleic acid is now washed and ready for elution. The filter is then connected to a elution reagent syringe and empty syringe. The elution reagent is passed across the filter to remove the nucleic acid from the filter and capture it in the elution reagent, which is then dispensed into a collection tube for analysis. The number of passes could be any number, such as but not limited to 1 to 1000 or more passes.

### Example 7

The number of passes to connected syringes can be any number. For example, the sample could be drawn into a syringe containing lysis and preservative reagent and then connected to the filter. Then a second syringe is connected to the other side of the filter. The lysis and preservative reagent is then pushed back and forth across the filter to capture the nucleic acid. The number of passes could be any number, such as but not limited to 1 to 1000 or more passes. The syringes are then disconnected. The filter is then connected to a wash reagent syringe and an empty syringe. The process of passing reagent across the filter is repeated. The number of passes could be any number, such as but not limited to 1 to 1000 or more passes. The nucleic acid is now washed and ready for elution. The filter is then connected to a elution reagent syringe and an empty syringe. The elution reagent is passed across the filter to remove the nucleic acid from the filter and capture it in the elution reagent, which is then dispensed into a collection tube for analysis. The number of passes could be any number, such as but not limited to 1 to 1000 or more passes.

### Example 8

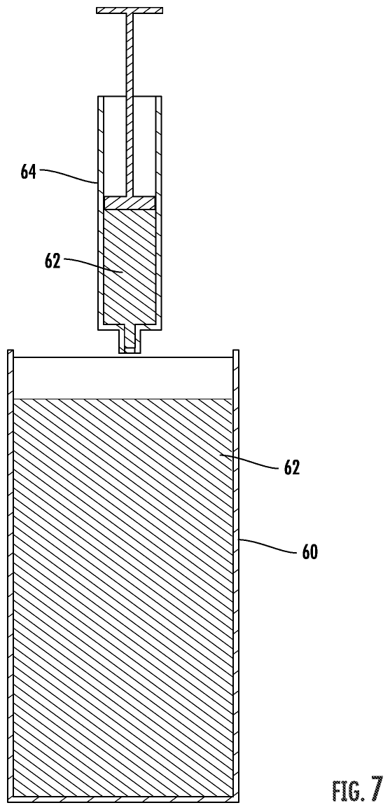

Referring to FIG. 7, a sample is collected by using a collection vessel 60 to put sample 62 in (i.e. water, blood, mixed fluids). Then a syringe 64 containing a lysis and preservative reagent, similar to the syringes 12 and 42, is used to draw some of the sample 62 into the syringe 64 for extraction. If the fluid is solid, it is first crushed or broken up and mixed with sterile water or a buffer solution. Then the contents are mixed thoroughly and the lysis syringe 64 is used to draw some of the sample up into the syringe 64.

It will be understood from the foregoing description that various modifications and changes may be made in the various embodiments of the present disclosure without departing from their true spirit. The description provided herein is intended for purposes of illustration only and is not intended to be construed in a limiting sense. Thus, while embodiments of the present disclosure have been described herein so that aspects thereof may be more fully understood and appreciated, it is not intended that the present disclosure be limited to these particular embodiments. On the contrary, it is intended that all alternatives, modifications and equivalents are included within the scope of the inventive concepts as defined herein. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulations and compositions described herein, the methods described herein or in the steps or the sequence of steps of the methods described herein without departing from the spirit and scope of the present disclosure.

What is claimed is:

1. A method of extracting nucleic acid from a sample, comprising:
(1) a first syringe containing a lysis and preservative reagent, (2) a second syringe containing an alcohol reagent, (3) a third syringe containing a quantity of nucleic acid-soluble, nuclease-free water, (4) a collection tube, and (5) a filtering device comprising a housing, an input end, an output end, and a nucleic acid-capturing filter securely positioned within the housing between the input end and the output end;
drawing the sample into the first syringe to mix the sample with the lysis and preservative reagent forming a liquid mixture containing cells, causing lysis of the cells of the sample and release of cellular nucleic acid therefrom;
attaching the input end of the filtering device to a needle end of the first syringe;
expressing the liquid mixture from the first syringe into the filtering device thereby causing the nucleic acid released from the cells to be captured on the filter as the liquid mixture passes through the filtering device;
separating the filtering device from the first syringe and connecting the input end of the filtering device to a needle end of the second syringe;
expressing the alcohol reagent from the second syringe into the filtering device thereby rinsing the nucleic acid and causing precipitation of the nucleic acid on the filter;
separating the filtering device from the second syringe and connecting the input end of the filtering device to a needle end of the empty second syringe;
expressing the air in the empty second syringe into the filtering device causing the filter to be dried of the alcohol reagent on the filter;
separating the filtering device from the empty second syringe and connecting the output end of the filtering device to a needle end of the third syringe;
connecting the collection tube to the input end of the filtering device; and
expressing the nucleic acid-soluble, nuclease-free water from the third syringe into the filtering device causing elution of the precipitated nucleic acid from the filter into the collection tube.

2. The method of claim 1, wherein a fourth syringe containing air and substantially absent of a liquid is provided.

3. The method of claim 2 further comprising the step of:
expressing the air in the fourth syringe into the filtering device causing evaporation of the alcohol reagent from the filter.

4. The method of claim 3 further comprising the step of:
separating the filtering device from the fourth syringe and connecting the output end of the filtering device to needle end of the third syringe.

5. The method of claim 1, wherein the lysis and preservative reagent is specific for preserving DNA.

6. The method of claim 1, wherein the lysis and preservative reagent is specific for preserving RNA.

7. A kit for extracting nucleic acid from a sample, the kit comprising:
a first syringe containing a lysis and preservative reagent;
at least one second syringe containing an alcohol reagent;
a third syringe containing a quantity of nucleic acid-soluble, nuclease-free water;
a collection tube; and
a filtering device comprising a housing, an input end, an output end, and a nucleic acid-capturing filter securely positioned within the housing between the input end and the output end, wherein the input end is connectable to at least the first syringe, the at least one second syringe, and the collection tube, and the output end is connectable at least to the third syringe.

8. The kit of claim 7 further comprising:
a fourth syringe containing air and substantially absent of a liquid.

9. The kit of claim 8, wherein the input end of the filtering device is connectable to the fourth syringe.

10. The kit of claim 7, wherein the lysis and preservative reagent is specific for preserving DNA.

11. The kit of claim 7, wherein the lysis and preservative reagent is specific for preserving RNA.

12. A system for extracting nucleic acid from a sample, the system comprising:
at least one first syringe containing a lysis and preservative reagent;
at least one second syringe containing an alcohol reagent;
at least one third syringe containing a quantity of nucleic acid-soluble, nuclease-free water;
at least one collection tube; and
at least one filtering device comprising a housing, an input end, an output end, and a nucleic acid-capturing filter securely positioned within the housing between the input end and the output end, wherein the input end is connectable to the at least one first syringe, the at least one second syringe, and the at least one collection tube, and the output end is connectable at least to the at least one third syringe.

13. The system of claim 12, further comprising:
at least one fourth syringe containing air and substantially absent of a liquid.

14. The system of claim 13, wherein the input end of the at least one filtering device is connectable to the at least one fourth syringe.

15. The system of claim 12, wherein the syringes and filter are used to lyse, wash and elute the nucleic acid.

16. The system of claim 12, wherein the system is used in field, outside of a laboratory setting.

17. The system of claim 12, wherein the lysis and preservative reagent is specific for preserving DNA.

18. The system of claim 12, wherein the lysis and preservative reagent is specific for preserving RNA.
